(19) United States
(12) Patent Application Publication
(10) Pub. No.: US 2017/0211103 A1
(43) Pub. Date: Jul. 27, 2017

(54) BIOSYNTHETIC PRODUCTION OF CHOLINE, ETHANOLAMINE, PHOSPHOETHANOLAMINE, AND PHOSPHOCHOLINE

(71) Applicant: 20n Labs, Inc., Washington, DC (US)

(72) Inventors: John Christopher Anderson, Berkeley, CA (US); Saurabh Srivastava, San Francisco, CA (US); Mark T. Daly, Oakland, CA (US)

(21) Appl. No.: 15/408,316

(22) Filed: Jan. 17, 2017

Related U.S. Application Data
(60) Provisional application No. 62/281,615, filed on Jan. 21, 2016.

(51) Int. Cl. C12P 13/00; C12N 9/16; C12N 9/10; C12N 9/88; C12N 9/12

(52) U.S. Cl. CPC C12P 13/001; C12N 9/88; C12N 9/1205; C12N 9/1007; C12N 9/16; C12Y 401/01065; C12Y 207/01082; C12Y 201/01103; C12Y 301/03075

(57) ABSTRACT

The present disclosure provides compositions and methods for the biosynthetic production of choline, ethanolamine, phosphoethanolamine, and phosphocholine.

### CROSS REFERENCE TO RELATED APPLICATIONS

This application claims priority to, and benefit of U.S. Provisional Application No. 62/281,615 filed Jan. 21, 2016, the contents of which are incorporated herein by reference in its entirety.

### INCORPORATION-BY-REFERENCE OF SEQUENCE LISTING

The contents of the text file named “NLAB_001_01US_ST25.txt” submitted electronically herewith which was created on Jan. 5, 2017 and is 20 KB in size, are incorporated herein by reference in their entirety.

### FIELD OF THE INVENTION

The present disclosure relates compositions and methods for the biosynthetic production of nutritional supplements such as choline and various intermediates and byproducts related to the production of choline. In particular, the disclosure features recombinant microorganisms comprising an engineered choline biosynthesis pathway.

### BACKGROUND OF THE INVENTION

Choline is a water-soluble nutrient usually grouped within the B-complex vitamins. Choline generally refers to the various quaternary ammonium salts containing the N,N,N-trimethylethanolammonium cation.

Some animals must consume choline through their diet to remain healthy. The most often available choline supplement is lecithin, derived from soy or egg yolks and often used as a food additive. Phosphatidylcholine is also available as a supplement, in pill or powder form. Supplementary choline is also available as choline chloride, which comes as a liquid due to its hydrophilic properties.

Choline is produced synthetically using various methods including one via trimethylamine and ethylene oxide (U.S. Pat. No. 3,373,201). It is noted that choline production is deceptively simple but can generate side products that affect taste and safety. Choline is a popular dietary supplement currently produced using chemical methods. In the laboratory choline can be prepared by methylation of dimethylethanolamine with methyl chloride. In the industrial Davy Process Technology route, choline chloride is produced from ethylene oxide, hydrochloric acid, and trimethylamine, or from the pre-formed salt.

A structurally and biologically-related chemical, ethanolamine, is used in the production of surfactants, polishes, pharmaceuticals, corrosion inhibitors, and chemical intermediates. Phosphoethanolamine and phosphocholine are potential dietary supplements.

Accordingly, there is a need in the an to produce choline and related structures or intermediates without possible disadvantageous side products.

### SUMMARY OF THE INVENTION

The present disclosure provides compositions and methods for the biosynthetic production of nutritional supplements such as choline as well as the production of intermediates of the choline synthesis pathway and/or structures related to choline.

Embodiments of the present invention comprise engineered organisms that produce choline. Embodiments of the present invention comprise engineered organisms that produce ethanolamine. Embodiments of the present invention comprise engineered organisms that produce phosphoethanolamine. Embodiments of the present invention comprise engineered organisms that produce phosphocholine. The engineered organisms may include genetically tractable organisms such as plants, animals, bacteria, or fungi.

The present invention comprises methods of producing choline. The methods comprise providing a recombinant microorganism comprising an engineered choline biosynthesis pathway. The engineered microorganism may be used for the commercial production of choline via fermentation. Accordingly, in one embodiment the invention provides growing in suitable conditions, a recombinant microbial host cell comprising at least one DNA molecule encoding an enzyme(s) that catalyze a substrate to product conversion selected from the group consisting of:

i. serine to ethanolamine (pathway step a);

ii. ethanolamine to phosphoethanolamine (pathway step b);

iii. phosphoethanolamine to phosphocholine (pathway step c);

iv. phosphocholine to choline (pathway step d);

wherein the at least one DNA molecule is heterologous to said microbial host cell and wherein said microbial host cell produces choline. The method further includes cultivating the microorganism in a culture medium until a recoverable quantity of choline is produced and recovering the choline.

In another embodiment, a biotransformation method of producing choline is provided. The method comprises providing a recombinant microorganism comprising an engineered choline biosynthesis pathway. The engineered microorganism may be used for the commercial production of choline. Accordingly, in one embodiment the invention provides growing in suitable conditions, a recombinant microbial host cell comprising at least one DNA molecule encoding an enzyme(s) that catalyze a substrate to product conversion selected from the group consisting of: i. ethanolamine to phosphoethanolamine (pathway step b); ii. phosphoethanolamine to phosphocholine (pathway step c); and iii. phosphocholine to choline e (pathway step d); wherein the at least one DNA molecule is heterologous to said microbial host cell, wherein ethanolamine substrate is added to the growth culture, and wherein said microbial host cell produces choline. The method further includes cultivating the microorganism in a culture medium until a recoverable quantity of choline is produced and recovering the choline.

The present invention comprises methods of producing ethanolamine. The methods comprise providing a recombinant microorganism comprising an engineered ethanolamine biosynthesis pathway. The engineered microorganism may be used for the commercial production of ethanolamine via fermentation. Accordingly, in one embodiment the invention provides growing in suitable conditions, a recombinant microbial host cell comprising at least one DNA molecule encoding an enzyme that catalyzes a serine to ethanolamine conversion, wherein the at least one DNA molecule is heterologous to said microbial host cell and wherein said microbial host cell produces ethanolamine. The method further includes cultivating the microorganism in a culture medium until a recoverable quantity of ethanolamine is produced and recovering the ethanolamine.

The present invention comprises methods of producing phosphoethanolamine. The methods comprise providing a recombinant microorganism comprising an engineered phosphoethanolamine biosynthesis pathway. The engineered microorganism may be used for the commercial production of phosphoethanolamine via fermentation. Accordingly, in one embodiment the invention provides growing in suitable conditions, a recombinant microbial host cell comprising at least one DNA molecule encoding an enzyme(s) that catalyze a substrate to product conversion selected from the group consisting of:

i. serine to ethanolamine (pathway step a);

ii. ethanolamine to phosphoethanolamine (pathway step b);

wherein the at least one DNA molecule is heterologous to said microbial host cell and wherein said microbial host cell produces phosphoethanolamine. The method further includes cultivating the microorganism in a culture medium until a recoverable quantity of phosphoethanolamine is produced and recovering the phosphoethanolamine.

In another embodiment, a biotransformation method of producing phosphoethanolamine is provided. The method comprises providing a recombinant microorganism comprising an engineered phosphoethanolamine biosynthesis pathway. The engineered microorganism may be used for the commercial production of phosphoethanolamine. Accordingly, in one embodiment the invention provides growing in suitable conditions, a recombinant microbial host cell comprising at least one DNA molecule encoding an enzyme that catalyzes the conversion of ethanolamine to phosphoethanolamine, wherein the at least one DNA molecule is heterologous to said microbial host cell, wherein ethanolamine substrate is added to the growth culture, and wherein said microbial host cell produces phosphoethanolamine. The method further includes cultivating the microorganism in a culture medium until a recoverable quantity of phosphoethanolamine is produced and recovering the phosphoethanolamine.

The present invention comprises methods of producing phosphocholine. The methods comprise providing a recombinant microorganism comprising an engineered phosphocholine biosynthesis pathway. The engineered microorganism may be used for the commercial production of phosphocholine via fermentation. Accordingly, in one embodiment the invention provides growing in suitable conditions, a recombinant microbial host cell comprising at least one DNA molecule encoding an enzyme(s) that catalyze a substrate to product conversion selected from the group consisting of:

i. serine to ethanolamine (pathway step a);

ii. ethanolamine to phosphoethanolamine (pathway step b);

iii. phosphoethanolamine to phosphocholine (pathway step c);

wherein the at least one DNA molecule is heterologous to said microbial host cell and wherein said microbial host cell produces phosphocholine. The method further includes cultivating the microorganism in a culture medium until a recoverable quantity of phosphocholine is produced and recovering the phosphocholine.

In another embodiment, a biotransformation method of producing phosphocholine is provided. The method comprises providing a recombinant microorganism comprising an engineered phosphocholine biosynthesis pathway. The engineered microorganism may be used for the commercial production of choline. Accordingly, in one embodiment the invention provides growing in suitable conditions, a recombinant microbial host cell comprising at least one DNA molecule encoding an enzyme(s) that catalyze a substrate to product conversion selected from the group consisting of: i. ethanolamine to phosphoethanolamine (pathway step b); ii. phosphoethanolamine to phosphocholine (pathway step c); and wherein the at least one DNA molecule is heterologous to said microbial host cell, wherein ethanolamine substrate is added to the growth culture, and wherein said microbial host cell produces phosphocholine. The method further includes cultivating the microorganism in a culture medium until a recoverable quantity of phosphocholine is produced and recovering the phosphocholine.

Some embodiments of the present invention comprise genetically engineered strains of yeast. In further embodiments, the yeast is S. cerevisiae. The present invention comprises engineered yeast strains that produce choline. Compositions of the present invention include yeast strains engineered with native and/or bacterial genes to produce choline from serine or ethanolamine. In certain embodiments, the engineered organisms encode enzymes that convert native serine metabolite to ethanolamine and then choline. In certain embodiments, the engineered organisms encode enzymes that produce ethanolamine by fermentation. In certain embodiments, the engineered organisms encode enzymes that produce phosphoethanolamine by fermentation. In certain embodiments, the engineered organisms encode enzymes that produce phosphocholine by fermentation.

In yet other embodiments, the engineered organisms encode enzymes that convert exogenously added ethanolamine to produce choline. In still other embodiments, the engineered organisms encode enzymes that convert exogenously added ethanolamine to produce phosphoethanolamine. In further embodiments, the engineered organisms encode enzymes that convert exogenously added ethanolamine to produce phosphocholine.

The yeast strains described herein can be used to produce the popular dietary supplement choline or intermediates in the synthesis pathway including ethanolamine, phosphoethanolamine, and phosphocholine. S. cerevisiae is a preferred organism for biosynthetic production due to favorable consumer sentiment, the robust experience and infrastructure for scaling up fermentation, and lack of potential phage infection.

In S. cerevisiae, choline is not produced as a free molecule, but phosphocholine is an essential intermediate in phosphatidylcholine production, and the genome encodes a choline kinase for salvage of extracellular choline. Therefore, in some aspects of the present invention, the engineered yeast have native choline kinase gene knocked out.

In one aspect, the engineered organisms have one to four genes or open reading frames under an inducible Gal promoter. The genes may be native to the host, heterologous, or a combination. In certain embodiments, the one to four genes are selected from the group consisting of AtSDC, Tbek1, AtNMT1, and PaPchP. The AtSDC gene encodes a serine decarboxylase that converts serine to ethanolamine (pathway step a). In some embodiments, ethanolamine is exogenously added to the growth culture and the serine conversion step is bypassed. Ethanolamine is subsequently phosphorylated to produce phosphoethanolamine (pathway step b). This may be achieved by an ethanolamine kinase encoded by the Tbek1 gene. Phosphoethanolamine is then methylated three times (pathway step c) to produce phosphocholine. This may be achieved by a phosphoethanolamine-N-methyltransferase encoded by the NMT1 gene. Phosphocholine is then dephosphorylated to choline (pathway step d). This may be achieved by phosphocholine phosphatase encoded by the PaPchP gene.

The yeast strains described herein can be used to produce the nutritional supplement choline from serine via fermentation. The yeast strains described herein can be used to produce ethanolamine, phosphoethanolamine, and/or phosphocholine via fermentation from sugars. The yeast strains described herein can be used to produce phosphoethanolamine, phosphocholine, and/or choline from exogenously added ethanolamine via biotransformation. The strains may be grown in a bioreactor and produce choline or intermediates of the synthesis pathway in the supernatant and/or cell pellet fraction. Subsequently, the product or intermediate can be purified. The strains encode enzymes that convert serine to ethanolamine, ethanolamine to phosphoethanolamine, phosphoethanolamine to phosphocholine, and/or phosphocholine to choline.

The present disclosure provides methods for the biosynthetic production of choline, ethanolamine, phosphoethanolamine, and phosphocholine. Embodiments of the present invention comprise growing engineered yeast strains using basic fermentation technologies. Using fermentation methods as opposed to chemical methods can bring the cost of the biological product as low as one-third the cost of the existing product, with additional benefits in reducing the capital costs of dedicated facilities, impact on the environment, safety of production workers, and potentially reduced impurities in the final product. The strains may be grown in a bioreactor and produce products in both the cell's cytoplasm and supernatant. Subsequently, the products can be purified and used as a dietary supplement or chemical product.

### BRIEF DESCRIPTION OF THE DRAWINGS

FIG. 1 shows the biosynthetic pathway encoded by strains of the present disclosure.

### DETAILED DESCRIPTION OF THE INVENTION

Unless otherwise indicated, the practice of the disclosure involves conventional techniques commonly used in molecular biology, microbiology, protein purification, protein engineering, protein and DNA sequencing, and recombinant DNA fields, which are within the skill of the art. Such techniques are known to those of skill in the art, and are described in numerous standard texts and reference works. All patents, patent applications, articles and publications mentioned herein, both supra and infra, are hereby expressly incorporated herein by reference.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art.

As used herein, the singular terms “a”, “an,” and “the” include the plural reference unless the context clearly indicates otherwise.

Unless otherwise indicated, nucleic acids are written left to right in 5′ to 3′ orientation and amino acid sequences are written left to right in amino to carboxyl orientation, respectively.

Numeric ranges are inclusive of the numbers defining the range. It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The headings provided herein are not limitations of the various aspects or embodiments of the invention which can be had by reference to the specification as a whole.

The term “invention” or “present invention” as used herein is a non-limiting term and is not intended to refer to any single embodiment of the particular invention but encompasses all possible embodiments as described in the specification and the claims.

A modified microorganism for high efficient production of choline and a variety of intermediates is provided herein. The present disclosure provides compositions and methods for an industrial fermentation process for the production of nutrient supplements such as choline. The fermentation is conducted using various species, including yeast, bacteria, and fungi. The microorganisms are genetically engineered to produce choline as well as intermediates such as ethanolamine, phosphoethanolamine, and phosphocholine.

The term “microorganism” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

“Bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include cocci, non-enteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

Yeasts are eukaryotic microorganisms classified as members of the fungus kingdom and are estimated to constitute 1% of all described fungal species. Yeasts are unicellular, although some species may also develop multicellular characteristics by forming strings of connected budding cells known as pseudo hyphae or false hyphae. Yeasts do not form a single taxonomic or phylogenetic grouping. The term “yeast” is often taken as a synonym for Saccharomyces cerevisiae, but the phylogenetic diversity of yeasts is shown by their placement in two separate phyla: the Ascomycota and the Basidiomycota.

The term “genus” is defined as a taxonomic group of related species according to the Taxonomic Outline of Bacteria and Archaea (Garrity, G. M., Lilburn, T. G., Cole, J. R., Harrison, S. H., Euzeby, J., and Tindall, B. J. (2007) The Taxonomic Outline of Bacteria and Archaea. TOBA Release 7.7, March 2007. Michigan State University Board of Trustees.

The term “species” is defined as a collection of closely related organisms with greater than 97% 16S ribosomal RNA sequence homology and greater than 70% genomic hybridization and sufficiently different from all other organisms so as to be recognized as a distinct unit.

As used herein, the term “isolated” when used in reference to a microbial organism is intended to mean an organism that is substantially free of at least one component as the referenced microbial organism is found in nature. The term includes a microbial organism that is removed from some or all components as it is found in its natural environment. The term also includes a microbial organism that is removed from some or all components as the microbial organism is found in non-naturally occurring environments. Therefore, an isolated microbial organism is partly or completely separated from other substances as it is found in nature or as it is grown, stored or subsisted in non-naturally occurring environments. Specific examples of isolated microbial organisms include partially pure microbes, substantially pure microbes and microbes cultured in a medium that is non-naturally occurring.

The term “gene” refers to a nucleic acid fragment that is capable of being expressed as a specific protein, optionally including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature.

The term “endogenous gene” refers to a native gene in its natural location in the genome of an organism.

A “foreign gene” or “heterologous gene” refers to a gene not normally found in the host organism, but that is introduced into the host organism by gene transfer. Foreign genes can comprise native genes inserted into a non-native organism, or chimeric genes.

A “transgene” is a gene that has been introduced into the genome by a transformation procedure.

As used herein, the term “open reading frame” also referred to as an “ORF” is the part of a reading frame that has the potential to code for a protein or peptide.

As used herein the term “coding sequence” refers to a DNA sequence that code for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure. As used herein the term “codon degeneracy” refers to the nature in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA.

The term “promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

As used herein, the term “genetically engineered” or “genetic engineering” or “genetic modification” involves the direct manipulation of an organism's genome using molecular and biotechnological tools and techniques. The present disclosure relates rational pathway design and assembly of biosynthetic genes, genes associated with operons, and control elements of such nucleic acid sequences, for the production of a desired metabolite, such as choline, in a microorganism.

As used herein, “metabolically engineered” can further include optimization of metabolic flux by regulation and optimization of transcription, translation, protein stability and protein functionality using genetic engineering and appropriate culture condition. The biosynthetic genes can be heterologous to the host (e.g., microorganism), either by virtue of being foreign to the host, or being modified by mutagenesis, recombination, or association with a heterologous expression control sequence in an endogenous host cell. Appropriate culture conditions are conditions such as culture medium pH, ionic strength, nutritive content, etc., temperature, oxygen, CO₂, nitrogen content, humidity, and other culture conditions that permit production of the compound by the host microorganism, i.e., by the metabolic action of the microorganism. Appropriate culture conditions are well known for microorganisms that can serve as host cells.

The term “recombinant microorganism” and “recombinant host cell” are used interchangeably herein and refer to microorganisms that have been genetically modified to express or over-express endogenous polynucleotides, or to express heterologous polynucleotides, such as those included in a vector, or which have an alteration in expression of an endogenous gene. By “alteration” it is meant that the expression of the gene, or level of a RNA molecule or equivalent RNA molecules encoding one or more polypeptides or polypeptide subunits, or activity of one or more polypeptides or polypeptide subunits is up regulated or down regulated, such that expression, level, or activity is greater than or less than that observed in the absence of the alteration. For example, the term “alter” can mean “inhibit,” but the use of the word “alter” is not limited to this definition.

The terms “metabolically engineered microorganism” and “modified microorganism” are used interchangeably herein and refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. The introduction of genetic material into a host or parental microorganism of choice modifies or alters the cellular physiology and biochemistry of the microorganism. Through the introduction of genetic material the parental microorganism acquires new properties, e.g. the ability to produce a new, or greater quantities of, an intracellular metabolite.

As used herein, the term “non-naturally occurring” when used in reference to a microbial organism or microorganism of the invention is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes or proteins within a choline biosynthetic pathway.

For example, the introduction of genetic material into a parental microorganism results in a new or modified ability to produce a chemical. The genetic material introduced into the parental microorganism contains gene, or parts of genes, coding for one or more of the enzymes involved in a biosynthetic pathway for the production of a chemical and may also include additional elements for the expression or regulation of expression of these genes, e.g. promoter sequences.

Those skilled in the art will understand that the genetic alterations, including metabolic modifications exemplified herein, are described with reference to a suitable host organism such as S. cerevisiae and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the S. cerevisiae metabolic alterations exemplified herein can readily be applied to other species by incorporating the same or analogous encoding nucleic acid from species other than the referenced species. Such genetic alterations include, for example, genetic alterations of species homologs, in general, and in particular, orthologues, paralogs or non-orthologous gene displacements.

An orthologue is a gene or genes that are related by vertical descent and are responsible for substantially the same or identical functions in different organisms. For example, mouse epoxide hydrolase and human epoxide hydrolase can be considered orthologues for the biological function of hydrolysis of epoxides. Genes are related by vertical descent when, for example, they share sequence similarity of sufficient amount to indicate they are homologous, or related by evolution from a common ancestor. Genes can also be considered orthologues if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity.

As used herein, the term “exogenous” or “heterologous” means that a biological function or material, including genetic material, of interest is not natural in a host strain. The term “native” means that such biological material or function naturally exists in the host strain or is found in a genome of a wild-type cell in the host strain.

Exogenous nucleic acid sequences involved in a pathway for production of choline can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

The term “expression” with respect to a gene sequence refers to transcription of the gene and, as appropriate, translation of the resulting mRNA transcript to a protein. Thus, as will be clear from the context, expression of a protein results from transcription and translation of the open reading frame sequence. The level of expression of a desired product in a host cell may be determined on the basis of either the amount of corresponding mRNA that is present in the cell, or the amount of the desired product encoded by the selected sequence. For example, mRNA transcribed from a selected sequence can be quantitated by PCR or by northern hybridization (see Sambrook et al., Molecular Cloning: A Laboratory Manual, Cold Spring Harbor Laboratory Press (1989)). Protein encoded by a selected sequence can be quantitated by various methods, e.g., by ELISA, by assaying for the biological activity of the protein, or by employing assays that are independent of such activity, such as western blotting or radioimmunoassay, using antibodies that are recognize and bind reacting the protein. See Sambrook et al., 1989, supra.

It is understood that the terms “recombinant microorganism” and “recombinant host cell” refer not only to the particular recombinant microorganism but to the progeny or potential progeny of such a microorganism. Because certain modifications may occur in succeeding generations due to either mutation or environmental influences, such progeny may not, in fact, be identical to the parent cell, but are still included within the scope of the term as used herein.

The term “wild-type microorganism” describes a cell that occurs in nature, i.e. a cell that has not been genetically modified. A wild-type microorganism can be genetically modified to express or overexpress a first target enzyme. This microorganism can act as a parental microorganism in the generation of a microorganism modified to express or overexpress a second target enzyme. In turn, the microorganism modified to express or overexpress a first and a second target enzyme can be modified to express or overexpress a third target enzyme.

Accordingly, a “parental microorganism” functions as a reference cell for successive genetic modification events. Each modification event can be accomplished by introducing a nucleic acid molecule in to the reference cell. The introduction facilitates the expression or overexpression of a target enzyme. It is understood that the term “facilitates” encompasses the activation of endogenous polynucleotides encoding a target enzyme through genetic modification of e.g., a promoter sequence in a parental microorganism. It is further understood that the term “facilitates” encompasses the introduction of heterologous polynucleotides encoding a target enzyme in to a parental microorganism.

As used herein the term “transformation” refers to the transfer of a nucleic acid fragment into a host organism, resulting in genetically stable inheritance. Host organisms containing the transformed nucleic acid fragments are referred to as “transgenic” or “recombinant” or “transformed” organisms.

The terms “plasmid”, “vector”, and “cassette” refer to an extra chromosomal element often carrying genes which are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitates transformation of a particular host cell. “Expression cassette” refers to a specific vector containing a foreign gene and having elements in addition to the foreign gene that allow for enhanced expression of that gene in a foreign host.

The term “protein,” “peptide,” or “polypeptide” as used herein indicates an organic polymer composed of two or more amino acidic monomers and/or analogs thereof. As used herein, the term “amino acid” or “amino acidic monomer” refers to any natural and/or synthetic amino acids including glycine and both D or L optical isomers. The term “amino acid analog” refers to an amino acid in which one or more individual atoms have been replaced, either with a different atom, or with a different functional group. Accordingly, the term polypeptide includes amino acidic polymer of any length including full length proteins, and peptides as well as analogs and fragments thereof. A polypeptide of three or more amino acids is also called a protein oligomer or oligopeptide

The term “enzyme” as used herein refers to any substance that catalyzes or promotes one or more chemical or biochemical reactions, which usually includes enzymes totally or partially composed of a polypeptide, but can include enzymes composed of a different molecule including polynucleotides.

As used herein, an “enzymatically active domain” refers to any polypeptide, naturally occurring or synthetically produced, capable of mediating, facilitating, or otherwise regulating a chemical reaction, without, itself, being permanently modified, altered, or destroyed. Binding sites (or domains), in which a polypeptide does not catalyze a chemical reaction, but merely forms noncovalent bonds with another molecule, are not enzymatically active domains as defined herein. In addition, catalytically active domains, in which the protein possessing the catalytic domain is modified, altered, or destroyed, are not enzymatically active domains as defined herein. Enzymatically active domains, therefore, are distinguishable from other (non-enzymatic) catalytic domains known in the art (e.g., detectable tags, signal peptides, allosteric domains, etc.).

The term “homolog”, used with respect to an original enzyme or gene of a first family or species, refers to distinct enzymes or genes of a second family or species which are determined by functional, structural or genomic analyses to be an enzyme or gene of the second family or species which corresponds to the original enzyme or gene of the first family or species. Most often, homologs will have functional, structural or genomic similarities. Techniques are known by which homologs of an enzyme or gene can readily be cloned using genetic probes and PCR. Identity of cloned sequences as homolog can be confirmed using functional assays and/or by genomic mapping of the genes.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. Thus, the term “homologous proteins” is defined to mean that the two proteins have similar amino acid sequences.

The term “analog” or “analogous” refers to nucleic acid or protein sequences or protein structures that are related to one another in function only and are not from common descent or do not share a common ancestral sequence. Analogs may differ in sequence but may share a similar structure, due to convergent evolution. For example, two enzymes are analogs or analogous if the enzymes catalyze the same reaction of conversion of a substrate to a product, are unrelated in sequence, and irrespective of whether the two enzymes are related in structure.

An expression vector or vectors can be constructed to include one or more choline biosynthetic pathway encoding nucleic acids as exemplified herein operably linked to expression control sequences functional in the host organism. Expression vectors applicable for use in the microbial host organisms of the invention include, for example, plasmids, phage vectors, viral vectors, episomes and artificial chromosomes, including vectors and selection sequences or markers operable for stable integration into a host chromosome.

Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art.

When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter.

The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

The term “fermentation” or “fermentation process” is defined as a process in which a microorganism is cultivated in a culture medium containing raw materials, such as feedstock and nutrients, wherein the microorganism converts raw materials, such as a feedstock, into products. Fermentation can be accomplished in batch or continuous production formats.

As used herein, the term “biotransformation” or “bioconversion” is the chemical modification made by an organism on a chemical compound.

As used interchangeably herein, the terms “activity” and “enzymatic activity” refer to any functional activity normally attributed to a selected polypeptide when produced under favorable conditions. Typically, the activity of a selected polypeptide encompasses the total enzymatic activity associated with the produced polypeptide. The polypeptide produced by a host cell and having enzymatic activity may be located in the intracellular space of the cell, cell-associated, secreted into the extracellular milieu, or a combination thereof.

As used herein, the term “choline biosynthesis” refers to the metabolic pathway that produces choline. The structure of choline is provided herein.

Choline may also be described with the following structure

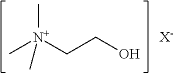

wherein X″ is a counter ion.

As used herein, the term “intermediates” refers to compounds or structures that are produced as part of the choline biosynthesis pathway described herein, and may themselves be final products. Intermediates include ethanolamine, phosphoethanolamine, and phosphocholine.

As used herein, the term “ethanolamine biosynthesis” refers to the metabolic pathway that produces ethanolamine. Ethanolamine may also be described with the following structure

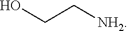

As used herein, the term “phosphoethanolamine biosynthesis” refers to the metabolic pathway that produces Phosphoethanolamine. Phosphoethanolamine may also be described with the following structure

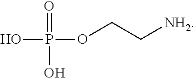

As used herein, the term “phosphocholine biosynthesis” refers to the metabolic pathway that produces phosphocholine. Phosphocholine may also be described with the following structure

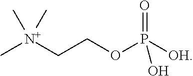

The term “serine decarboxylase” refers to an enzyme that catalyzes the conversion of serine to ethanolamine. These enzymes are available from a vast array of organisms. The enzyme may be, for example, encoded by the AtSDC gene from Arabidopsis thaliana.

The term “ethanolamine kinase” refers to an enzyme that catalyzes the conversion of ethanolamine to phosphoethanolamine. These enzymes are available from a vast array of organisms. The enzyme may be, for example, encoded by the Tbek1 gene from Trypanosoma brucei.

The term “phosphoethanolamine N-methyltransferase” refers to an enzyme that catalyzes the conversion of phosphoethanolamine to phosphocholine. These enzymes are available from a vast array of organisms. The enzyme may be, for example, encoded by the AtNMT1 gene from Arabidopsis thaliana.

The term “phosphocholine phosphatase” refers to an enzyme that catalyzes the conversion of phosphocholine to choline. These enzymes are available from a vast array of organisms. The enzyme may be, for example, encoded by the PaPchP gene from Pseudomonas aeruginosa.

The first step (pathway step a) in choline biosynthesis is the direct decarboxylation of serine to ethanolamine which is catalyzed by a serine decarboxylase. Ethanolamine is recognized as the entrance compound to choline biosynthesis. Therefore, this step can by bypassed by the direct addition of ethanolamine to the culture medium.

In the second step (pathway step b), ethanolamine is phosphorylated by ethanolamine kinase to produce phosphoethanolamine.

In the next step (pathway step c), phosphoethanolamine is methylated three times by phosphoethanolamine N-methyltransferase to produce phosphocholine.

In the final step (pathway step d), phosphocholine is dephosphorylated by phosphocholine phosphatase to yield free choline.

The term “volumetric productivity” or “production rate” is defined as the amount of product formed per volume of medium per unit of time. Volumetric productivity is reported in gram per liter per hour (g/L/h).

The term “yield” is defined as the amount of product obtained per unit weight of raw material and may be expressed as g product per g substrate (g/g). Yield may be expressed as a percentage of the theoretical yield. “Theoretical yield” is defined as the maximum amount of product that can be generated per a given amount of substrate as dictated by the stoichiometry of the metabolic pathway used to make the product.

The term “titer” is defined as the concentration of a substance in solution. Herein, it also refers to the concentration of product, usually expressed in g/L, upon completion of fermentation.

### Construction of Production Host

Recombinant organisms containing the necessary genes that will encode the enzymatic pathway for the biosynthetic production of choline may be constructed using techniques well known in the art. In the present invention, genes encoding the enzymes of one of the choline biosynthetic pathways of the invention, for example, serine decarboxylase, ethanolamine kinase, phosphoethanolamine N-methyltransferase, and phosphocholine phosphatase, may be determined from the genomes of various organisms, as described above.

Methods of obtaining desired genes from a genome are common and well known in the art of molecular biology. For example, if the sequence of the gene is known, suitable synthetic genes are constructed by gene synthesis. Tools for codon optimization for expression in a heterologous host are readily available.

Once the relevant pathway genes are identified, the synthesized genes may be assembled into larger genetic constructs such as into suitable vectors. Means for this are well known in the art. Vectors or cassettes useful for the transformation of a variety of host cells are common and commercially available from gene synthesis companies such as DNA2.0, SGI-DNA, Invitrogen, and Genscript. Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. Both control regions may be derived from genes homologous to the transformed host cell, although it is to be understood that such control regions may also be derived from genes that are not native to the specific species chosen as a production host.

### Engineered Microorganisms

According to one embodiment, a modified microorganism comprising a heterologous production system of choline is provided. The modified microorganisms may be yeast, bacteria, or fungi. The modified microorganisms may express heterologous proteins useful in the production of choline or its intermediates. The modified microorganism may have native host genes knocked out from the host genome to enhance production of choline.

One embodiment of the present invention is a non-naturally occurring microorganism having a choline pathway and comprising at least four open reading frames encoding choline pathway enzymes expressed in a sufficient amount to produce choline, wherein said choline pathway comprises (i) an enzyme that converts serine to ethanolamine, (ii) an enzyme that converts ethanolamine to phosphoethanolamine, (iii) enzyme that converts phosphoethanolamine to phosphocholine, and (iv) an enzyme that converts phosphocholine to choline.

Another embodiment of the present invention is a non-naturally occurring microorganism having a choline pathway and comprising at least three open reading frames encoding choline pathway enzymes expressed in a sufficient amount to produce choline, wherein said choline pathway comprises (i) an enzyme that converts ethanolamine to phosphoethanolamine, (ii) enzyme that converts phosphoethanolamine to phosphocholine, and (iii) an enzyme that converts phosphocholine to choline.

One embodiment of the present invention is a non-naturally occurring microorganism comprising a biosynthesis pathway and at least three open reading frames encoding enzymes expressed in a sufficient amount to produce phosphocholine, wherein said pathway comprises (i) an enzyme that converts serine to ethanolamine, (ii) an enzyme that converts ethanolamine to phosphoethanolamine, and (iii) enzyme that converts phosphoethanolamine to phosphocholine.

Another embodiment of the present invention is a non-naturally occurring microorganism having a biosynthesis pathway and comprising at least two open reading frames encoding enzymes expressed in a sufficient amount to produce phosphocholine, wherein said pathway comprises (i) an enzyme that converts ethanolamine to phosphoethanolamine and (ii) an enzyme that converts phosphoethanolamine to phosphocholine.

One embodiment of the present invention is a non-naturally occurring microorganism having a biosynthesis pathway and comprising at least one open reading frame encoding enzymes expressed in a sufficient amount to produce phosphoethanolamine, wherein said pathway comprises (i) an enzyme that converts serine to ethanolamine, and (ii) an enzyme that converts ethanolamine to phosphoethanolamine.

Yet another embodiment of the present invention is a non-naturally occurring microorganism having a biosynthesis pathway and comprising at least one open reading frame encoding an enzyme expressed in a sufficient amount to produce phosphoethanolamine, wherein said enzyme converts ethanolamine to phosphoethanolamine.

One embodiment of the present invention is a non-naturally occurring microorganism comprising a biosynthesis pathway and at least one open reading frame encoding an enzyme expressed in a sufficient amount to produce ethanolamine, wherein said pathway comprises an enzyme that converts serine to ethanolamine.

In some embodiments of the present invention, the enzyme that converts serine to ethanolamine is a serine decarboxylase. In other embodiments of the present invention, the enzyme that converts ethanolamine to phosphoethanolamine is an ethanolamine kinase. In certain embodiments of the present invention, the enzyme that converts phosphoethanolamine to phosphocholine is a phosphoethanolamine N-methyltransferase. In further embodiments of the present invention, the enzyme that converts phosphocholine to choline is a phosphocholine phosphatase.

Examples of heterologous genes that may be expressed in modified microorganisms of the present invention include genes that encode enzymes such as serine decarboxylase, phosphoethanolamine kinase, phosphoethanolamine N-methyl transferase, and phosphocholine phosphatase. These genes may be derived from animals, plants, bacteria, yeast, or fungi. Serine decarboxylase genes include but are not limited to AtSDC from Arabidopsis thaliana. Phosphoethanolamine kinase genes include but are not limited to Tbek1 from Trypanosoma brucei. Phosphoethanolamine N-methyl transferase genes include but are not limited to AtNMT1 from Arabidopsis thaliana. Phosphocholine phosphatase genes include but are not limited to PaPchP from Pseudomonas aeruginosa. Further, said nucleic acid encoding molecules (e.g., genes) may be codon optimized for use in an organism of interest.

In some embodiments, the modified microorganism is a yeast cell. In some embodiments, the recombinant microorganisms may be yeast recombinant microorganisms of the Saccharomyces clade. In certain embodiments, the modified yeast may be Saccharomyces cerevisiae. The S. cerevisiae may be strain S288C or a derivative thereof.

The modified yeast may encode at least one heterologous gene selected from the group consisting of phosphocholine phosphatase, phosphoethanolamine N-methyltransferase, ethanolamine kinase, and serine decarboxylase. The heterologous genes may be derived from bacteria, yeast, fungi, plants, or animals. The serine decarboxylase may be an Arabidopsis thaliana AtSDC gene and encode a polypeptide comprising SEQ ID NO: 1 or the active domain thereof. The ethanolamine kinase may be a Trypanosoma brucei Tbek1 gene and encode a polypeptide comprising SEQ ID NO: 2 or the active domain thereof. The phosphoethanolamine N-methyltransferase may be an Arabidopsis thaliana AtNM1 gene and encode a polypeptide comprising SEQ ID NO: 3 or the active domain thereof. The phosphocholine phosphatase may be a Pseudomonas aeruginosa PaPchP gene and encode a polypeptide comprising SEQ ID NO: 4 or the active domain thereof.

In some embodiments, the modified yeast may have native choline kinase gene knocked out. The native enzyme may result in a futile cycle of phosphorylation/dephosphorylation. Therefore, by knocking out the choline kinase gene and hence, the enzyme, the dephosphorylation of phosphocholine is pushed forward in the chemical pathway and limits the reverse reaction (phosphorylation of choline to phosphocholine). For instance, the modified yeast may have the CKI1 choline kinase gene deleted wherein the gene encodes a polypeptide comprising SEQ ID NO: 5.

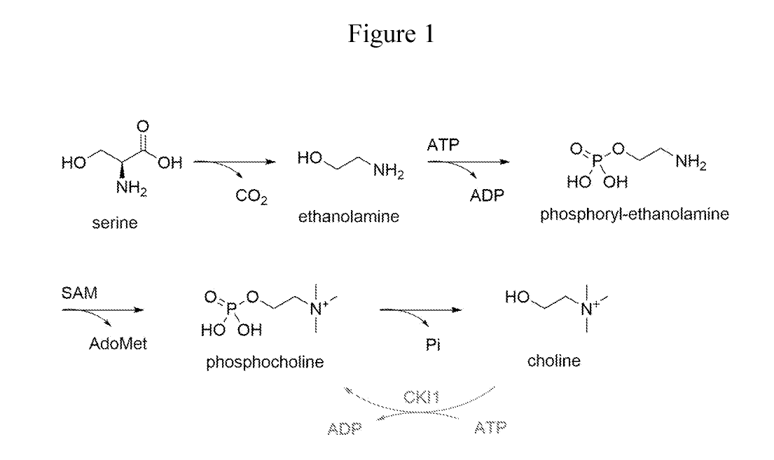

The biosynthetic pathway encoded by these strains is described in FIG. 1. The amino acid serine is decarboxylated to ethanolamine via the action of serine decarboxylase. Ethanolamine is then phosphorylated by ethanolamine kinase, methylated thrice by phosphoethanolamine N-methyltransferase, and then dephosphorylated by phosphocholine phosphatase to yield free choline. Strain ch1 differs from ch2 by the knockout of the CKI1 gene encoding choline kinase (FIG. 1), This native enzyme would result in a futile cycle of phosphorylation/dephosphorylation of choline in our design. Indeed, a higher production of choline was observed in strain ch1 compared to ch2. When grown in SC Minimal Broth with 2% raffinose and 1% galactose, strains cal produces choline in supernatant and in cell pellets, as indicated by LC-MS analysis.

### Methods of Production

The present disclosure provides methods for the biosynthetic production of choline and intermediates such as ethanolamine, phosphoethanolamine, and phosphocholine using engineered microorganisms of the present invention.

In one embodiment, a method of producing choline via fermentation is provided. The method comprises providing a fermentation media comprising carbon substrate; contacting said media with a recombinant yeast microorganism expressing an engineered choline biosynthetic pathway wherein said pathway comprises the following substrate to product conversions: i. serine to ethanolamine (pathway step a); ii. ethanolamine to phosphoethanolamine (pathway step b); iii. phosphoethanolamine to phosphocholine (pathway step c); and iv. phosphocholine to choline (pathway step d); and culturing the yeast in conditions whereby choline is produced. In methods of the present inventions, the substrate to product conversion of pathway step “a” is performed by a serine decarboxylase; the substrate to product conversion of pathway step “b” is performed by an ethanolamine kinase; the substrate to product conversion of pathway step “c” is performed by a phosphoethanolamine N-methyl transferase; and, the substrate to product conversion of pathway step “d” is performed by a phosphocholine phosphatase. The method further includes cultivating the microorganism in a culture medium until a recoverable quantity of choline is produced and recovering the choline.

In another embodiment, a method of producing choline via biotransformation is provided. The method comprises providing a media comprising carbon substrate and exogenously added ethanolamine; contacting said media with a recombinant yeast microorganism expressing an engineered choline biosynthetic pathway wherein said pathway comprises the following substrate to product conversions: i. ethanolamine to phosphoethanolamine (pathway step b); ii. phosphoethanolamine to phosphocholine (pathway step c); and iii. phosphocholine to choline (pathway step d); and culturing the yeast in conditions whereby choline is produced. In methods of the present inventions, the substrate to product conversion of pathway step “b” is performed by an ethanolamine kinase; the substrate to product conversion of pathway step “c” is performed by a phosphoethanolamine N-methyl transferase; and, the substrate to product conversion of step “d” is performed by a phosphocholine phosphatase. The method further includes cultivating the microorganism in a culture medium until a recoverable quantity of choline is produced and recovering the choline.

In one embodiment, a method of producing phosphocholine via fermentation is provided. The method comprises providing a fermentation media comprising carbon substrate; contacting said media with a recombinant yeast microorganism expressing an engineered biosynthetic pathway wherein said pathway comprises the following substrate to product conversions: i. serine to ethanolamine (pathway step a); ii. ethanolamine to phosphoethanolamine (pathway step b) and; iii. phosphoethanolamine to phosphocholine (pathway step c); and culturing the yeast in conditions whereby choline is produced. In methods of the present inventions, the substrate to product conversion of pathway step “a” is performed by a serine decarboxylase; the substrate to product conversion of pathway step “b” is performed by an ethanolamine kinase; the substrate to product conversion of pathway step “c” is performed by a phosphoethanolamine N-methyl transferase. The method further includes cultivating the microorganism in a culture medium until a recoverable quantity of phosphocholine is produced and recovering the phosphocholine.

In another embodiment, a method of producing phosphocholine via biotransformation is provided. The method comprises providing a media comprising carbon substrate and exogenously added ethanolamine; contacting said media with a recombinant yeast microorganism expressing an engineered biosynthesis pathway wherein said pathway comprises the following substrate to product conversions: i. ethanolamine to phosphoethanolamine (pathway step b); and ii. phosphoethanolamine to phosphocholine (pathway step c); and culturing the yeast in conditions whereby phosphocholine is produced. In methods of the present inventions, the substrate to product conversion of pathway step “b” is performed by an ethanolamine kinase and the substrate to product conversion of pathway step “c” is performed by a phosphoethanolamine N-methyl transferase. The method further includes cultivating the microorganism in a culture medium until a recoverable quantity of phosphocholine is produced and recovering the phosphocholine.

In one embodiment, a method of producing phosphoethanolamine via fermentation is provided. The method comprises providing a fermentation media comprising a carbon substrate; contacting said media with a recombinant yeast microorganism expressing an engineered biosynthetic pathway wherein said pathway comprises: i. serine to ethanolamine (pathway step a); and ii. ethanolamine to phosphoethanolamine (pathway step b); and culturing the yeast in conditions whereby phosphoethanolamine is produced. In methods of the present inventions, the substrate to product conversion of pathway step “a” is performed by a serine decarboxylase and the substrate to product conversion of pathway step “b” is performed by an ethanolamine kinase. The method further includes cultivating the microorganism in a culture medium until a recoverable quantity of phosphoethanolamine is produced and recovering the phosphoethanolamine.

In another embodiment, a method of producing phosphoethanolamine via biotransformation is provided. The method comprises providing a media comprising carbon substrate and exogenously added ethanolamine; contacting said media with a recombinant yeast microorganism expressing an enzyme that converts ethanolamine to phosphoethanolamine, and culturing the yeast in conditions whereby phosphoethanolamine is produced. In methods of the present inventions, the ethanolamine to phosphoethanolamine is performed by an ethanolamine kinase. The method further includes cultivating the microorganism in a culture medium until a recoverable quantity of phosphoethanolamine is produced and recovering the phosphoethanolamine.

In one embodiment, a method of producing ethanolamine via fermentation is provided. The method comprises providing a fermentation media comprising a carbon substrate; contacting said media with a recombinant yeast microorganism expressing an enzyme that convers serine to ethanolamine; and culturing the yeast in conditions whereby ethanolamine is produced. In methods of the present inventions, the serine to ethanolamine conversion is performed by a serine decarboxylase. The method further includes cultivating the microorganism in a culture medium until a recoverable quantity of ethanolamine is produced and recovering the ethanolamine.

Some embodiments of the present invention comprise yeast strains (designated ch1 and ch2) derived from S. cerevisiae strain S288C Each encodes at least 3 foreign genes under inducible Gal promoters. Strain ch1 also contains a single gene knockout. The specific proteins encoded by each strain and their sequences, source, and accession numbers are provided in Table 1. The genes for these proteins are synthesized with yeast-optimized codon usage, assembled into singular genetic cassettes, and then inserted into the HO or CKI1 locus of S288C under URA2 selection.

Serine and the cofactors involved in the choline pathway are universal to all organisms, and thus the host organism could be any genetically tractable organism (plants, animals, bacteria, or fungi). Amongst yeasts, other species such as S. pombe or P. pastoris are plausible alternatives. Within the S. cerevisiae species, other strains more amenable to large-scale productions, such as CENPalpha, may be utilized.

The Gal promoter used in embodiments of the present invention could be replaced with constitutive promoters, or other chemically-inducible, growth phase-dependent, or stress-induced promoters. Heterologous genes of the present invention may be genomically encoded or alternatively encoded on plasmids or yeast artificial chromosomes (YACs). All genes introduced could be encoded with alternate codon usage without altering the biochemical composition of the system. All enzymes used in embodiments of the present invention have extensive orthologues in the biosphere that could be encoded as alternatives.

### Culture Conditions

The growth medium used to test for production of choline by the engineered strains was Teknova SC Minimal Broth with Raffinose minus Uracil supplemented with 1% galactose.

A variety of purification protocols including solid phase extraction and cation exchange chromatography may be employed to purify the desired products from the culture supernatant or the yeast cell pellet fraction.

### Examples

### Example 1: Strain Development

Two functioning yeast strains, ch1 and ch2, were derived from S. cerevisiae strain S288C (Table 1) and constructed with the following features: a serine decarboxylase gene (AtSDC) derived from Arabidopsis thaliana (Accession number Q9MA74) capable of expressing a polypeptide comprising SEQ ID NO:1, an ethanolamine kinase gene (Tbek1) derived from Trypanosoma brucei (Accession number CAP73998) capable of expressing a polypeptide comprising SEQ. ID NO: 2, a phosphoethanolamine N-methyltransferase gene (AtNMT1) derived from Arabidopsis thaliana (Accession number AAG41121) capable of expressing a polypeptide comprising SEQ II) NO: 3, and a phosphocholine phosphatase gene (PaPchP) derived from Pseudomonas aeruginosa (Accession number Q9HTR2) capable of expressing a polypeptide comprising SEQ ID NO: 4. Strain cal is further characterized in that the native choline kinase gene (CKI1) Accession number AAA34499 capable of expressing a polypeptide comprising SEQ ID NO: 5 is knocked out, impaired, or non-functional.

TABLE 1
Strain constructs

| Strain | Accession No. | Source | Name | Enzyme |
|---|---|---|---|---|
| ch1/ch2 | Q9HTR2 | Pseudomonas | PaPchP | phosphocholine |
| | | aeruginosa | | phosphatase |
| | AAG41121 | Arabidopsis thaliana | AtNMT1 | phosphoethanolamine |
| | | | | N-methyltransferase |
| | CAP73998 | Trypanosoma brucei | Tbek1 | ethanolamine kinase |
| | Q9MA74 | Arabidopsis thaliana | AtSDC | serine decarboxylase |
| ch1 | AAA34499 | (native knockout) | CKI1 | choline kinase |

The biosynthetic pathway encoded by these strains is described in FIG. 1. The AtSDC gene from Arabidopsis thaliana encodes serine decarboxylase that decarboxylates serine to ethanolamine. This step may be bypassed by exogenous addition of ethanolamine. Ethanolamine is subsequently phosphorylated by ethanolamine kinase encoded by the Tbek1 gene from Trypanosoma brucei to produce phosphoryl ethanolamine. This product is methylated three times by phosphoethanolamine N-methyltransferase encoded by the AtNMT1 from Arabidopsis thaliana. The final step is the dephosphorylation of phosphocholine by phosphocholine phosphatase, encoded by the PaPchP gene from Pseudomonas aeruginosa. In a strain in which native choline kinase is knocked out, such as strain ch1, the futile phosphorylation/dephosphorylation cycle of choline is disrupted.

Strains were grown in SC Minimal Broth with 2% raffinose and 1% galactose. The growth medium used to test for production of choline by the engineered strains was Teknova SC Minimal Broth with Raffinose minus Uracil supplemented with 1% galactose. Data collected for production of choline by strains ch1 and ch2 was performed in 4 mL cultures grown in 24-well blocks for 2 days at 30° C. with shaking.

1. A modified microorganism for production of choline comprising at least three heterologous enzymes selected from the group consisting of serine decarboxylase, ethanolamine kinase, phosphoethanolamine N-methyltransferase, and phosphocholine phosphatase.

2. The modified microorganism of claim 1 wherein the serine decarboxylase comprises SEQ ID NO: 1 or the active domain thereof.

3. The modified microorganism of claim 1 wherein the ethanolamine kinase comprises SEQ ID NO: 2 or the active domain thereof.

4. The modified microorganism of claim 1 wherein the phosphoethanolamine N-methyltransferase comprises SEQ ID NO: 3 or the active domain thereof.

5. The modified microorganism of claim 1 wherein the phosphocholine phosphatase comprises SEQ ID NO: 4 or the active domain thereof.

6. The modified microorganism of claim 1, wherein the modified microorganism is yeast or bacteria.

7. The modified microorganism of claim 6, wherein the yeast is Saccharomyces cerevisiae strain S288C.

8. The modified microorganism of claim 1 wherein a native choline kinase gene is knocked out.

9. A method of producing choline comprising: (a) culturing the cells of claim 1 under suitable conditions for the production of choline; (b) producing choline; and (c) recovering choline.

10. The method of claim 9 wherein recovering the choline comprises collecting the choline from yeast cells, supernatant, or the combination thereof.

11. A non-naturally occurring microbial organism comprising at least three heterologous genes encoding choline pathway enzymes expressed in a sufficient amount to produce choline, wherein said choline pathway comprises (i) an enzyme that converts serine to ethanolamine, (ii) an enzyme that converts ethanolamine to phosphoethanolamine, (iii) enzyme that converts phosphoethanolamine to phosphocholine, and (iv) an enzyme that converts phosphocholine to choline.

12. The non-naturally occurring microbial organism of claim 11 wherein said enzyme that converts serine to ethanolamine is a serine decarboxylase, wherein said enzyme that converts ethanolamine to phosphoethanolamine is an ethanolamine kinase, wherein said enzyme that converts phosphoethanolamine to phosphocholine is a phosphoethanolamine N-methyltransferase, and wherein said enzyme that converts phosphocholine to choline is a phosphocholine phosphatase.

13. The non-naturally occurring microbial organism of claim 12 wherein said serine decarboxylase comprises SEQ ID NO: 1, said ethanolamine kinase comprises SEQ ID NO: 2, phosphoethanolamine N-methyltransferase comprises SEQ ID NO: 3, and said phosphocholine phosphatase comprises SEQ ID NO: 4.

14.-29. (canceled)


SEQUENCE LISTING (5 sequences)

SEQ ID NO: 1 - protein, 482 aa, Arabidopsis thaliana
MVGSLESDQTLSMATLIEKLDILSDDFDPTAVVTEPLPPPVTNGIGADKGGGGGEREMVLGRNIHTTSLAVTEPEVNDEFTGDKEAYMASVLARYRKTLVERTKNHLGYPYNLDFDYGALGQLQHFSINNLGDPFIESNYGVHSRPFEVGVLDWFARLWEIERDDYWGYITNCGTEGNLHGILVGREMFPDGILYASRESHYSVFKAARMYRMECEKVDTLMSGEIDCDDLRKKLLANKDKPAILNVNIGTTVKGAVDDLDLVIKTLEECGFSHDRFYIHCDGALFGLMMPFVKRAPKVTFNKPIGSVSVSGHKFVGCPMPCGVQITRMEHIKVLSSNVEYLASRDATIMGSRNGHAPLFLWYTLNRKGYKGFQKEVQKCLRNAHYLKDRLREAGISAMLNELSSTVVFERPKDEEFVRRWQLACQGDIAHVVVMPSVTIEKLDNFLKDLVKHRLIWYEDGSQPPCLASEVGTNNCICPAHK

SEQ ID NO: 2 - protein, 431 aa, Trypanosoma brucei
MEVAVGHVRHVPCTVPDDPASRMRAIREIVARHCALFMQKGGDVGRSQCASNLGNNITSGTSNGFDADRLQVVAFSGGITNEMYHVYCEGWEAHSVVMRIFGKETERVISRGDELFYQSLFIPTYVRGQNFLVYQYLYQYEPLPFVEMVKEYEKIARGIAFFHVRATLEARSDYSVTPVGGVVGAGGATVGPTNTCSRFDLEENYIVHSLTEWVEQALSETVQEKVDATKRATYASVAQQLKEEAQWLLPLMQRHSPELGESTCHNDLLSGNIMRQKSDGALKIIDFEYAKRNYFLFDIANHFNEYTGLECDYATYFPSEEHMKKFVTTYMCAMREELEKHAEEAKRRQLTDIIPGQQHFFMCDVGGVEGDKRIHRMVQLVKLLTLASHLSWSVWALLQEAVSKTDMDFLRYSQLRLSRYLETRKEFSASS

SEQ ID NO: 3 - protein, 491 aa, Arabidopsis thaliana
MAASYEEERDIQKNYWIEHSADLTVEAMMLDSRASDLDKEERPEVLSLLPPYEGKSVLELGAGIGRFTGELAQKAGELIALDFIDNVIKKNESINGHYKNVKFMCADVTSPDLKITDGSLDLIFSNWLLMYLSDKEVELLAERMVGWIKVGGYIFFRESCFHQSGDSKRKSNPTHYREPRFYSKVFQECQTRDAAGNSFELSMIGCKCIGAYVKNKKNQNQICWIWQKVSSENDRGFQRFLDNVQYKSSGILRYERVFGQGFVSTGGLETTKEFVEKMNLKPGQKVLDVGCGIGGGDFYMAEKFDVHVVGIDLSVNMISFALERAIGLSCSVEFEVADCTTKHYPDNSFDVIYSRDTILHIQDKPALFRTFFKWLKPGGKVLISDYCRSPKTPSAEFSEYIKQRGYDLHDVQAYGQMLKDAGFTDVIAEDRTDQFMQVLKRELDRVEKEKEKFISDFSKEDYDDIVGGWKSKLERCASDEQKWGLFIANKN

SEQ ID NO: 4 - protein, 349 aa, Pseudomonas aeruginosa
MTFAKGILAALALAAAVGQASATELEHWPAPAARQLNALIEANANKGAYAVFDMDNTSYRYDLEESLLPYLEMKGVLTRDRLDPSLKLIPFKDQAGHKESLFSYYYRLCEIDDMVCYPWVAQVFSGFTLRELKGYVDELMAYGKPIPATYYDGDKLATLDVEPPRVFSGQRELYNKLMENGIEVYVISAAHEELVRMVAADPRYGYNAKPENVIGVTTLLKNRKTGELTTARKQIAEGKYDPKANLDLEVTPYLWTPATWMAGKQAAILTYIDRWKRPILVAGDTPDSDGYMLFNGTAENGVHLWVNRKAKYMEQINGMIKQHSAAQAKAGLPVTADRNWVIVTPEQIQ

SEQ ID NO: 5 - protein, 582 aa, Saccharomyces cerevisiae
MVQESRPGSVRSYSVGYQARSRSSSQRRHSLTRQRSSQRLIRTISIESDVSNITDDDDLRAVNEGVAGVQLDVSETANKGPRRASATDVTDSLGSTSSEYIEIPFVKETLDASLPSDYLKQDILNLIQSLKISKWYNNKKIQPVAQDMNLVKISGAMTNAIFKVEYPKLPSLLLRIYGPNIDNIIDREYELQILARLSLKNIGPSLYGCFVNGRFEQFLENSKTLTKDDIRNWKNSQRIARRMKELHVGVPLLSSERKNGSACWQKINQWLRTIEKVDQWVGDPKNIENSLLCENWSKFMDIVDRYHKWLISQEQGIEQVNKNLIFCHNDAQYGNLLFTAPVMNTPSLYTAPSSTSLTSQSSSLFPSSSNVIVDDIINPPKQEQSQDSKLVVIDFEYAGANPAAYDLANHLSEWMYDYNNAKAPHQCHADRYPDKEQVLNFLYSYVSHLRGGAKEPIDEEVQRLYKSIIQWRPTVQLFWSLWAILQSGKLEKKEASTAITREEIGPNGKKYIIKTEPESPEEDFVENDDEPEAGVSIDTFDYMAYGRDKIAVFWGDLIGLGIITEEECKNFSSFKFLDTSYL